(19) United States
(12) Patent Application Publication
(10) Pub. No.: US 2006/0140869 A1
(43) Pub. Date: Jun. 29, 2006

(54) Polymeric contrast agents for use in medical imaging

(76) Inventors: Egidijus Uzgiris, Niskayuna, NY (US); Mohan Amaratunga, Clifton Park, NY (US); Brian Grimmond, Clifton Park, NY (US); Daniel Meyer, Rexford, NY (US); Joanne Smith, Ballston Lake, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/022,984

(22) Filed: Dec. 23, 2004

(51) Int. Cl. A61K 49/14

(52) U.S. Cl. 424/9.34; 530/400

(57) ABSTRACT

A contrast agent comprising a polypeptide is provided. The polypeptide contains lysine residues and optionally, one or more types of amino acid residues selected from the group consisting of glutamic acid residues and aspartic acid residues, wherein the lysine residues are substituted with a group derived from a steric hindrance molecule; and an image producing entity is present in a range between about 100 units and about 2000 units. Methods for administering the aforementioned contrast agent are also provided.

### BACKGROUND

1. Technical field

The present disclosure relates to improved conjugated polymers for medical treatment of tumor tissue, and more specifically, for optimizing drug delivery to tumor tissue as well as to the diagnostic imaging of tumors.

2. Description of Related Art

In many medical procedures it is important to accumulate a certain active agent to a desired tissue type. For example, in chemotherapy, it is important to deliver drugs only to cancerous tumor tissue, and not to normal tissue, since these drugs destroy the tissue with which they come in contact. Another example would be in medical imaging. Contrast agents are attached to carrier molecules which are specific to tumor tissue. As the carrier molecules concentrate in the tumor tissue, the contrast agents enhance a medical image of this tissue.

One known type of carrier molecule contains polypeptides having a diameter larger than pores of blood vessels of normal tissue and smaller than pores of blood vessels of tumor tissue. See U.S. Pat. No. 5,762,909. These carriers have a length several orders of magnitude greater than their diameter, a net negative charge, and form a worm-like chain conformation with a long persistence length. Lanthanide complexes (e.g., gadolinium-diethylenetriamine pentaacetic acid complexes) are attached to these carrier molecules to create complex molecules which are introduced into a blood vessel of the subject.

These complex molecules pass though the pores of only the tumor endothelium and interact with the fibrous structures of the tumor interstitium. The penetration of the tumor interstitium by the complex molecules is enhanced by the worm-like configuration of the complex molecule which allows the molecule to “snake” around fixed obstacles in the extracellular matrix of the tumor interstitium.

Steric hindrance molecules such as DTPA can chelate with image producing entities such as lanthanum series and more specifically, gadolinium ions. In magnetic resonance imaging (MRI), the image producing entities produce a detectable change in the proton relaxivity of water in tissue. Unfortunately, the signal change that is required for robust detection necessitates a large number of image producing entities attached to the polypeptide. Additionally, the greater the number of image producing entities requires a larger polypeptide structure which cannot translocate across the vasculature to enter the tissue of interest.

Accordingly, there remains a need to provide polypeptide-DTPA molecules that provide the desired conformation that has the capability of carrying a large number of image producing entities.

### SUMMARY

The present invention provides a contrast agent comprising a polypeptide containing lysine residues and optionally, one or more types of amino acid residues selected from the group consisting of glutamic acid residues and aspartic acid residues, wherein the lysine residues are substituted with a group derived from a steric hindrance molecule; and

an image producing entity present in a range between about 100 ions and about 2000 ions.

The present invention further provides a method comprising

a) administering a contrast agent to a subject wherein the contrast agent comprises a polypeptide containing lysine residues and optionally, one or more types of amino acid residues selected from the group consisting of glutamic acid residues and aspartic acid residues, wherein the lysine residues are substituted with a group derived from a steric hindrance molecule and an image producing entity present in a range between about 100 ions and about 2000 ions; and

b) imaging the subject via magnetic resonance imaging.

In yet another embodiment, the present invention provides a method of pre-targeting tissue comprising:

a) injecting an antibody labeled with a receptor molecule into a subject;

b) injecting a contrast agent to the subject wherein the contrast agent comprises a polypeptide containing lysine residues and optionally, one or more types of amino acid residues selected from the group consisting of glutamic acid residues and aspartic acid residues, wherein the lysine residues are substituted with a group derived from a steric hindrance molecule, an image producing entity present in a range between about 100 ions and about 2000 ions, and a targeting ligand wherein the targeting ligand binds to the receptor molecule; and

c) imaging the subject via magnetic resonance imaging.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawing, in which:

FIG. 1 are a series of images taken of a Mat B tumor wherein the imaging was done pre-injection of a Polylysine-Gd-DTPA reptating polymer, one minute after injection, 60 minutes after injection, and 24 hours after injection.

FIG. 2 is a histogram of signal response in a Mat B tumor post injection of a Polylysine-Gd-DTPA reptating polymer.

### DETAILED DESCRIPTION OF THE INVENTION

The present invention provides a contrast agent with enhanced magnetic resonance signals. The contrast agent has a polymeric backbone of polypeptides (herein also referred to as poly(amino acids)). The polypeptides are substituted with steric hindrance molecules and an image producing entity which is sufficient to enhance magnetic resonance signals. The image producing entity is present in a range between about 100 ions and about 2000 ions, and preferably in a range between about 500 ions and about 1500 ions. With this high number of imagine producing entities, the amplification factor for magnetic resonance signals per binding event is sufficiently high to detect 104 receptors per cell at a 50% cell density in tissue. The expected signal is calculated to be a factor of 1.5 over the minimum detection threshold (AS/S minimum is about 0.1 in good signal to noise 1.5 Tesla imaging systems). At a field of 3 Tesla, the signal increases by another factor of 2 due to the higher field. Hence, the polypeptides of the present invention with the high number of image producing entities are sufficiently effective in MRI to allow detection of a range of diseased tissue with specific targeted interactions.

The nature of the polymer backbone of the polymeric contrast agent is not critical, provided that the polymer has pendant groups which can be reacted with activated steric hindrance molecules (“SHM”) as described below to provide a polymer having an elongated structure. Suitable pendant groups which may be present in the polymer include amine groups which form homo- and co-polymers of poly(amino acids). Poly(amino acids) contain two or more amino acids. Preferably, the polypeptide is selected from the group consisting of polylysine, polyglutamic acid, polyaspartic acid, and a copolymer of lysine and either glutamic acid or aspartic acid. More preferably, the polypeptide is polylysine. Other polymers may be used provided that after reaction with the SHM, the resulting polymer has an elongated structure characterized by a molecular length that is in a range between about 5 and about 500 times the cross-sectional diameter of the polymer molecule and a net negative charge in an aqueous environment. In addition, the polymer preferably is of sufficient length to increase the time in which the product circulates in the blood. For polypeptides, the polymer backbone can advantageously be in a range between about 35 and about 1500 amino acid residues long and is preferably in a range between about 100 and about 800 amino acid residues long. The length of the polymeric contrast agents in the present invention allows the polymeric contrast agents to translocate across the tumor endothelium with high efficiency. A process called reptation allows elongagted worm-like molecules of the present invention to wiggle around obstacles, and to pass through restricted openings that globular or coiled molecules would be unable to pass through. Additionally, the polymeric contrast agents of the present invention do not rapidly migrate to the extravascular space of the tumor before being excreted through the kidneys. Because the polymeric backbone is synthetic, the length can be tailored to provide desired resistance times in the body. Clearance from the blood is rapid for short molecules, resulting in a short plasma lifetime. Plasma lifetime increases rapidly as the polymers increase in length. For example, where the polymer is a polypeptide, a plateau is reached for a molecular length of about 500 residues and little further change in lifetime occurs. Not only does the use of a synthetic polypeptide provide the ability to modify the polymer length so as to change the blood circulations times to smaller values, but the ability to modify the polymer length to probe small permeability modulations is also provided.

The polypeptide may be a random copolymer which contains lysine units and either glutamic acid units, aspartic acid units, or both. Glutamic and/or aspartic acid units may constitute from about 20 to about 60 percent of the copolymer. Particularly useful copolymers have glu:lys ratio of about 1:4 to about 6:4. A high content of lysine is believed advantageous for imaging as it allows a high loading of the copolymer with paramagnetic ions. Without wishing to be bound by any theory, it is believed that the presence of glutamic acid residues in the copolymer backbone accomplishes two things. First, it is believed that the glutamic acid residues provide a stiffer initial copolymer backbone for the synthesis of the complete construct. Second, it is believed that the presence of glutamic acid residues in the copolymer promotes extension of the final polymer through charge repulsion. Suitable copolymers can be synthesized using techniques known to those skilled in the art. Suitable copolymers are also commercially available from a variety of sources.

At least a portion of the lysine groups of the polymeric contrast agent have a steric hindrance molecule (“SHM”) attached thereto. The SHM is any molecule that by its physical size enforces an elongated conformation by providing steric hindrance between neighboring steric hindrance molecules. Preferably the SHM presents negative charges in an aqueous environment along the polymer chain to assist in keeping the polymer backbone straight through coulombic repulsion. A net negative charge for each SHM creates charge repulsion between adjacent SHM which assists in the polymer backbone retaining its elongated, worm-like configuration.

Particularly preferred steric hindrance molecules are molecules that chelate with paramagnetic entities. As those skilled in the art will appreciate, paramagnetic entities include certain transition metals and lanthanide ions. Any molecule known to complex with paramagnetic entities and which is of sufficient size to provide steric hindrance against polymer bending can be used as the SHM. Preferably, the group present on the polymer backbone that is derived from the SHM exhibits a net negative charge in an aqueous environment. Suitable lanthanide ion chelating molecules include, but are not limited to diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis[3-(4-carboxyl)-butanoic acid], 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA). Ligands useful for chelating for other ions (such as, for example, Fe(m), Mn(II), Cu(II), etc.) include bis(thiosemicarbazone) and derivatives, porphyrins and derivatives, 2,3-Bis(2-thioacetamido)propionates and derivatives, N,N′-bis(mercaptoacetyl)-2,3-diaminopropanoate, and bis(aminoethanethiol) and derivatives. The preferred SHM is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA).

In accordance with the present invention, the SHM contains or chelates an image producing entity. Suitable image producing entities include paramagnetic entities and entities which undergo nuclear reaction to emit a particle, such as, for example, an alpha particle, a gamma particle, a beta particle, or a positron. Such imaging entities are known to those skilled in the art. Gamma emitters include, for example, ¹¹¹In and ¹⁵³Gd. Positron emitters include, for example, ⁸⁹Zr, which may be employed in positron emission tomography (PET) imaging. Preferably, the image producing entity is ¹⁵³Gd.

Typically, to attach the SHM to the polymer backbone, an activating group is provided on the SHM. The activating group present on the SHM can be any group which will react with the polymer. Suitable groups include, but are not limited to, mixed carbonate carbonic anhydride groups, amine groups, succinimidyl groups and dicyclohexylcarbodiimide (DCC) groups. Those skilled in the art will readily envision reaction schemes for attaching an activating group to any given SHM.

In particularly preferred methods, a substantially mono-activated steric hindrance molecule (“SHM”) is provided. The term “activated” means that a functional group is provided on the SHM which permits covalent bonding of the molecule to the copolymer chain. By the term “substantially mono-activated” it is meant that about 90% or more of the steric hindrance molecules contain only a single activated site.

The precise conditions for reacting the polymer with the substantially mono-activated SHM will depend upon a number of factors including the particular polymer chosen and the specific SHM used. Those skilled in the art will readily envision reaction schemes for any given pair of materials to produce the desired polymer-SHM conjugates.

In an exemplary embodiment of the present invention, the polymers are made by substituting the lysine residues of polylysine with DTPA in a mixed anhydride reaction (as disclosed in U.S. patent application Ser. No. 10/209,726 entitled, “Synthesis of Highly Conjugated Polymers”, which is incorporated herein by reference). In order to attain the reptating conformation, the anhydride reaction and the coupling reaction is preferably run at a temperature in a range between about −25° C. and about −28° C. for 30 minutes under dry nitrogen atmosphere. The coupling of the anhydride to the lysines is modified in that a much higher molar ratio of anhydride to lysines residues is used in the coupling (from 7 to 10). After the coupling reaction, the reaction solution is subject to roto-vaporation at 50° C. to release all the volatile organic molecules and then the produce is purified through extensive dia-filtration (Amicon, 10 kD molecular weight cutoff filters). To achieve the final MR active agent, the paramagnetic ion gadolinium is incorporated into the product polymer chelating DTPA groups. By way of example, a paramagnetic ion such as gadolinium can be loaded into chelating DTPA groups by dropwise addition of a gadolinium salt such as, for example, gadolinium chloride or gadolinium citrate in 0.1 M HCl (50 mM in Gd) into the polymer solution (15 mM NaHCO3). The dropwise addition of Gd continues until a slight indication of free Gd (not chelated by available DTPA groups) is noted (small aliquots of polymer solution added to 10 microMolar of arzenzo IIII in acetate buffer—free Gd turns the dye solution blue). The Gd-loaded highly conjugated polypeptide is then ready for introduction into a blood vessel of the subject.

In certain embodiments of the present invention, the conjugated polymer can be used for drug delivery. It is contemplated, for example, that the SHM can itself be a therapeutic agent. It is also contemplated that a therapeutic agent can be attached at a few sites along the substituted polymer chain. By way of example, chemotherapeutic agents (such as, for example, doxorubicin or methotrexate) which have been shown to have activity against tumors can be attached to the conjugated polymer. Even though specific chemotherapy drugs are mentioned here, any known chemotherapy drugs capable of being attached to the specific polypeptide being used may be employed. Also, plant and bacterial toxins such as ricin and abrin and the like may be used. For therapy, one could alternatively use a radiotherapeutic agent such as ⁹⁰Y or ²¹¹At.

The therapeutic entity can be attached to the conjugated copolymer using techniques known to those skilled in the art. It is also contemplated that therapeutic agents can be used in combination with other types of active agents incorporated into the conjugated copolymer. For example, the polymer backbone can be highly conjugated with a non-therapeutic SHM which chelates an image producing entity and a therapeutic agent can appear at only a few sites along the backbone. As another example, the copolymer backbone can be highly conjugated with a non-therapeutic SHM, and a therapeutic agent can be bound to the SHM, rather than being bound directly to the copolymer backbone.

In other embodiments, the conjugated polymer can be used for targeting specific tissue. It is contemplated, for example, that the SHM can itself be a targeting agent. It is also contemplated that a targeting ligand can be attached at a few sites along the substituted polymer chain. The targeting ligand can be attached to the conjugated polymer using techniques known to those skilled in the art. It is also contemplated that targeting agents can be used in combination with other types of active agents incorporated into the conjugated polymer. For example, the polymer backbone can be highly conjugated with a non-targeting SHM which chelates an image producing entity and a targeting ligand can appear at only a few sites along the backbone. As another example, the polymer backbone can be highly conjugated with a non-targeting SHM, and a targeting agent can be bound to the SHM, rather than being bound directly to the polymer backbone. For instance, small molecules such as biotin and folic acid can be used to target specific tissue.

In another embodiment of the present invention, an antibody is injected into a subject prior to the administration of the polymeric agent. The antibody is linked with a receptor protein that binds with a targeting ligand such as biotin or folic acid. The protein can be any protein known in the art that binds with the chosen targeting molecules. Typically, the protein is avidin when the targeting molecule is biotin. Upon injection of the polymeric agent that contains the targeting ligand, the polymeric agent translocates into the tumor to find the antibody with the receptor protein. Subsequently, the targeting agent and protein attach. For instance, the avidin protein binds strongly to the biotin small molecule.

The high net negative charge for the contrast agents is desirable since it also assists in the contrast agents to retaining their elongated, worm-like conformation. Since many in vivo molecules tend to have a negative charge, it is advantageous for the contrast agents to also have a net negative charge in order to avoid agglutination with blood plasma proteins. Positively charged molecules are also known to stick to cell surfaces (which are generally negatively charged).

In order to perform one preferred method of using the present compositions, a subject is first imaged and then a polymeric contrast agent in accordance with this disclosure is introduced into the subject by injecting the contrast agent intravenously. The dose of the polymeric contrast agent can be in the range of about 0.01 mmoles Gd/Kg to about 0.1 mmoles Gd/Kg. The subject is then imaged at one or more pre-selected tissue sites. The subject is imaged, preferably beginning immediately after injection and at certain timed intervals. Preferably, the timed intervals are shortly after injection (within 10 minutes) and up to 1 hour post injection. An image at 24 hours may also be acquired.

The following examples are included for purposes of illustrating certain aspects of the subject matter disclosed herein and should not be interpreted as limiting the scope of the overall disclosure herein.

### EXAMPLE 1

An animal model was used to demonstrate the MR imaging effects associated with angiogenesis. Fisher female rats were implanted subcutaneously with rat mammary adenocarcinoma cells (ATTC Mat B cells) that were grown to a suitable density in tissue culture. The implanted cells grew into tumors of 1-2 cm diameter in about 10 to 14 days and continued to grow to larger sizes when experiments extended beyond that time frame.

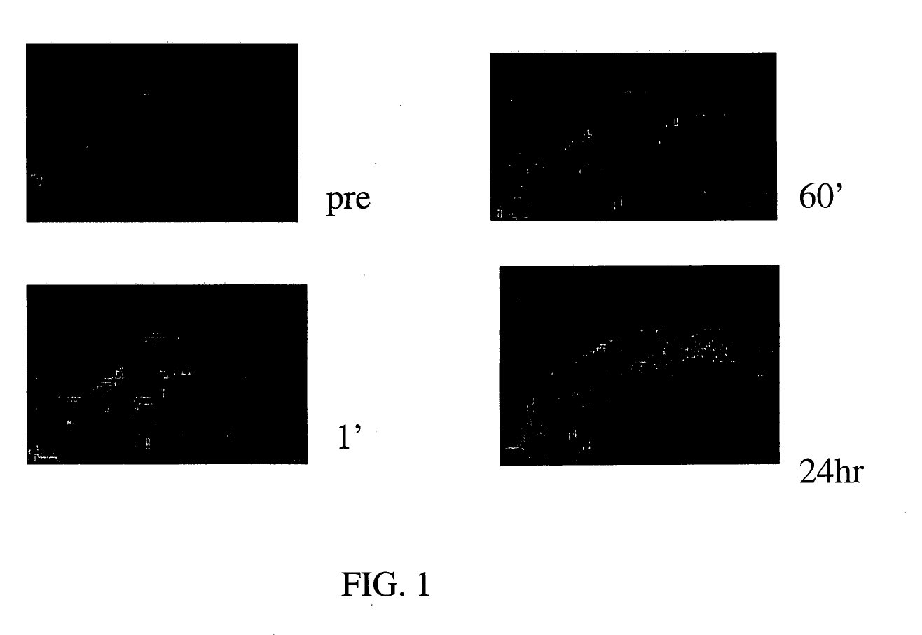

The reptating polymer that was used (Polylysine-Gd-DTPA) was synthesized using a synthesis method described above with polymer length of 780 monomer units, molecular weight of 460 kDaltons, and about 98% of lysines acylated with DTPA (i.e. about 764 Gd ions). The animals were injected intravenously at a dose of 0.025 mmoles Gd/kG. FIG. 1 are a series of images taken wherein the imaging was done pre-injection, one minute after injection, 60 minutes after injection, and 24 hours after injection with a T1 weighted spin echo pulse sequence (TR=250 ms, TE=9 ms), 12 centimeter FOV, 1 millimeter slices. Receive coil was a solenoid coil of about 5 centimeters diameter. Imaging was done on a Signa 1.5 Tesla scanner.

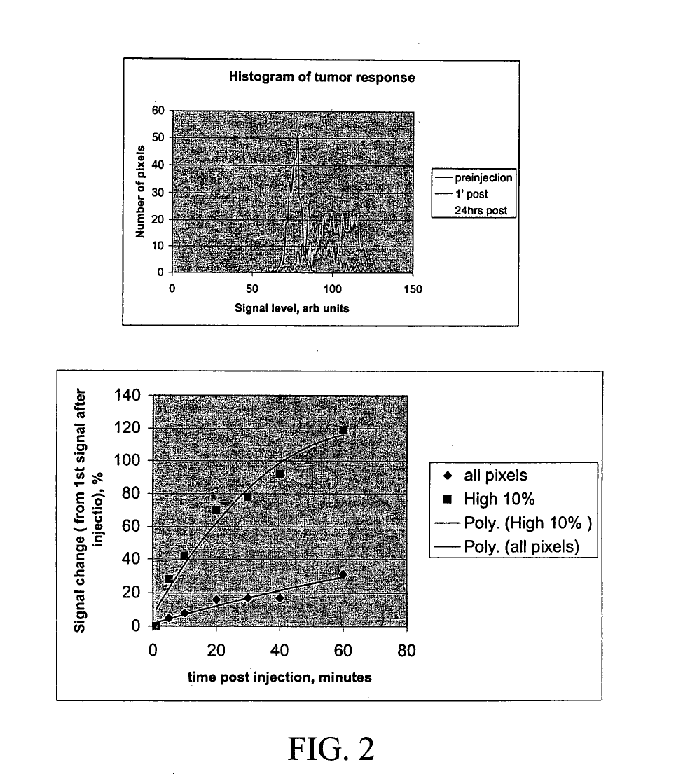

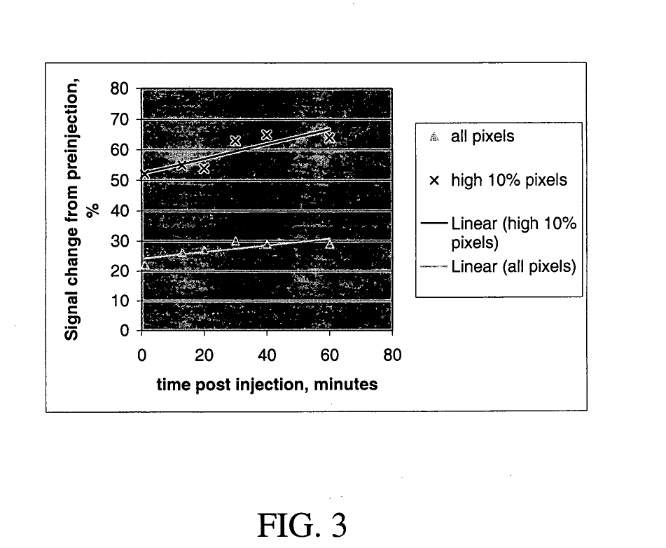

FIG. 2 is is a graph of the slopes in signal change for all the pixels in the tumor and the highest 10% of pixels in the tumor. The signals are heterogenous in space due to angiogenesis being most prevalent in the outer rim of the tumor. Thus in order to capture the regions of interest with high permeability, the average signal associated with the pixels of the entire tumor in the image slice is plotted in addition to the average signal associated with the highest 10% of the pixels. The highest 10% of the pixels represents the hypermeable regions associated with angiogenic processes. The use of all pixels represents the average values for the entire tumor which includes then the center of tumors which are often necrotic and unreactive in terms of blood perfusion. These regions are not biologically relevant and thus the total average values have more “noise” that are associated with the necrotic processes which are not of direct interest in the present case.

### EXAMPLE 2

In this example the polymer was not in an extended conformation due to a lower conjugation that promoted a coiled configuration. The polymer that was used (Polylysine-Gd-DTPA) had a mean polymer length of 402 monomer units with about 85% of lysines acylated with DTPA (i.e. about 342 Gd ions). The coiled polymer was injected into Fisher female rats as described above. FIG. 3 is a graph of the slopes in signal change for all the pixels in the tumor and the highest 10% of pixels in the tumor. Calculation of the slope via linear regression fits gave a 7% per hour signal change for all the pixels in the tumor and a 14% per hour signal change for the highest 10% of the pixels in the tumor.

When comparing the signal change of the reptating polymer of Example 1 and the polymer of Example 2, it is evident that the reptating polymer with the greater number of Gd ions gave enhanced magnetic resonance signals.

In addition to tumor visualization, the polypeptide agents in accordance with the present disclosure can advantageously be used in other applications. With relatively good blood clearance properties, the present intravascular polymeric agents may be useful for angiography. They also do not appear to accumulate in other organs such as muscle, kidney or liver. Therefore, the present agents may be preferred for drug delivery/imaging over others that are based on globular proteins or coiled homopolymers, which tend to show accumulation in liver and kidneys of animal models.

While specific embodiments of the invention have been illustrated and described herein, it is realized that modifications and changes will occur to those skilled in the art. It is therefore to be understood that the appended claims are intended to cover all such modifications and changes as fall within the true spirit and scope of the invention.

What we claim is:

1. A contrast agent comprising a polypeptide containing lysine residues and optionally, one or more types of amino acid residues selected from the group consisting of glutamic acid residues and aspartic acid residues, wherein the lysine residues are substituted with a group derived from a steric hindrance molecule; and
an image producing entity present in a range between about 100 ions and about 2000 ions.

2. The contrast agent in accordance with claim 1, wherein the image producing entity is a paramagnetic entity.

3. The contrast agent in accordance with claim 2, wherein the paramagnetic entity is gadolinium ions.

4. The contrast agent in accordance with claim 1, wherein the polypeptide comprises gadolinium ions in a range between about 500 ions and about 1500 ions.

5. The contrast agent in accordance with claim 1, wherein the steric hindrance molecule is selected from the group consisting of diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis(3-(4-carboxyl)-butanoic acid), 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA).

6. The contrast agent in accordance with claim 5, wherein the steric hindrance molecule is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA).

7. The contrast agent in accordance with claim 1, wherein the polypeptide is a homopolymer of lysine.

8. The contrast agent in accordance with claim 1, wherein the polypeptide is a random copolymer of lysine and glutamic acid.

9. The contrast agent in accordance with claim 1, further comprising a targeting ligand.

10. The contrast agent in accordance with claim 9, wherein the targeting ligand comprises at least one molecule selected from the group consisting of biotin and folic acid.

11. The contrast agent in accordance with claim 10, wherein the targeting ligand is biotin.

12. A contrast agent comprising a polylysine, wherein the lysine residues are substituted with a group derived from a steric hindrance molecule; and
an image producing entity of gadolinium ions present in a range between about 100 ions and about 2000 ions.

13. A method comprising
a) administering a contrast agent to a subject wherein the contrast agent comprises a polypeptide containing lysine residues and optionally, one or more types of amino acid residues selected from the group consisting of glutamic acid residues and aspartic acid residues, wherein the lysine residues are substituted with a group derived from a steric hindrance molecule and an image producing entity present in a range between about 100 ions and about 2000 ions; and
b) imaging the subject via magnetic resonance imaging.

14. The method in accordance with claim 13, wherein the step of administering a contrast agent includes a contrast agent further comprising a targeting ligand.

15. The method in accordance with claim 14, wherein the targeting ligand comprises at least one molecule selected from the group consisting of biotin and folic acid.

16. The method in accordance with claim 15, wherein the therapeutic ligand is biotin.

17. The method in accordance with claim 14, wherein the subject is exposed to an antibody prior to administering the contrast agent wherein the antibody is labeled with a receptor molecule that binds the targeting ligand.

18. The method in accordance with claim 17, wherein the receptor molecule is avidin.

19. The method in accordance with claim 13, wherein the polypeptide is a homopolymer of lysine.

20. The method in accordance with claim 13, wherein the image producing entity is a paramagnetic entity.

21. The method in accordance with claim 20, wherein the paramagnetic entity is gadolinium ions.

22. The method in accordance with claim 21, wherein the polypeptide comprises gadolinium ions in a range between about 500 ions and about 1500 ions.

23. The method in accordance with claim 13, wherein the steric hindrance molecule is selected from the group consisting of diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis(3-(4-carboxyl)-butanoic acid), 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA).

24. The method in accordance with claim 23, wherein the steric hindrance molecule is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA).

25. The method in accordance with claim 13, wherein the contrast agent is administered at a dose in the range of 0.01 mmoles Gd/Kg to about 0.1 mmoles Gd/Kg.

26. A method of pre-targeting tissue comprising:
a) injecting an antibody labeled with a receptor molecule into a subject;
b) injecting a contrast agent to the subject wherein the contrast agent comprises a polypeptide containing lysine residues and optionally, one or more types of amino acid residues selected from the group consisting of glutamic acid residues and aspartic acid residues, wherein the lysine residues are substituted with a group derived from a steric hindrance molecule, an image producing entity present in a range between about 100 ions and about 2000 ions, and a targeting ligand wherein the targeting ligand binds to the receptor molecule; and
c) imaging the subject via magnetic resonance imaging.

27. The method in accordance with claim 26, wherein the receptor molecule is avidin.

28. The method in accordance with claim 26, wherein the targeting ligand comprises at least one molecule selected from the group consisting of biotin and folic acid

29. The method in accordance with claim 28, wherein the targeting ligand is biotin.

30. The method in accordance with claim 26, wherein the polypeptide is a homopolymer of lysine.

31. The method in accordance with claim 26, wherein the image producing entity is a paramagnetic entity.

32. The method in accordance with claim 31, wherein the paramagnetic entity is gadolinium ions.

33. The method in accordance with claim 32, wherein the polypeptide comprises gadolinium ions in a range between about 500 ions and about 1500 ions.

34. The method in accordance with claim 26, wherein the steric hindrance molecule is selected from the group consisting of diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis(3-(4-carboxyl)-butanoic acid), 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA).

35. The method in accordance with claim 34, wherein the steric hindrance molecule is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA).

36. The method in accordance with claim 26, wherein the contrast agent is administered at a dose in the range of 0.01 mmoles Gd/Kg to about 0.1 mmoles Gd/Kg.
